(19) United States
(12) Patent Application Publication
(10) Pub. No.: US 2015/0037408 A1
(43) Pub. Date: Feb. 5, 2015

(54) Delayed Release Pharmaceutical Compositions of Salsalate

(71) Applicants: Kothari Jay Shantilal, Ahmedabad (IN); Muthaiyyan Esakkimuthu Kannan, Ahmedabad (IN); Mistry Gaurav Navinbhai, Ahmedabad (IN); Brij Khera, Princeton, NJ (US)

(72) Inventors: Kothari Jay Shantilal, Ahmedabad (IN); Muthaiyyan Esakkimuthu Kannan, Ahmedabad (IN); Mistry Gaurav Navinbhai, Ahmedabad (IN); Brij Khera, Princeton, NJ (US)

(21) Appl. No.: 14/520,329

(22) Filed: Oct. 21, 2014

Related U.S. Application Data
(63) Continuation-in-part of application No. PCT/IN2013/000273, filed on Apr. 23, 2013.
(60) Provisional application No. 61/894,277, filed on Oct. 22, 2013.

(30) Foreign Application Priority Data
Apr. 23, 2012 (IN) 1277/MUM/2012

(51) Int. Cl. A61K 31/618; B65B 7/16; A61K 9/48; A61K 9/50; A61K 47/38; A61K 9/28

(52) U.S. Cl. CPC A61K 31/618; A61K 47/38; A61K 9/2866; A61K 9/4891; A61K 9/5047; A61K 9/2893; A61K 9/5089; B65B 7/16
USPC 424/465; 560/71; 514/159; 424/490; 427/2.21; 264/330; 53/467

(57) ABSTRACT

The present invention relates to modified release pharmaceutical compositions comprising salsalate. The invention also relates to processes for the preparation of such compositions.

### CROSS-REFERENCE TO RELATED APPLICATIONS

This application claims priority to U.S. Provisional Patent Application No. 61/894,277, filed on Oct. 22, 2013 and priority as a continuation-in-part of PCT/IN2013/000273, filed on Apr. 23, 2013, which designates the United States and claims priority from Indian Provisional Patent Application No. 1277/MUM/2012, filed on Apr. 23, 2012. The contents of each of these applications are incorporated herein in their entirety by reference.

### FIELD OF THE INVENTION

The present invention relates to modified release pharmaceutical compositions comprising salsalate. The invention also relates to processes for the preparation of such compositions.

### BACKGROUND OF THE INVENTION

Salsalate (salicylsalicylic acid; 2-hydroxybenzoic acid 2-carboxyphenyl ester) is a nonsteroidal anti-inflammatory drug (NSAID) having a structure of Formula I.

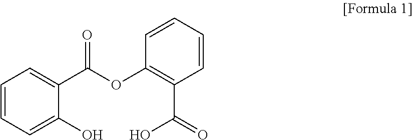 [SMILES: O=C(Oc1ccccc1C(=O)O)c1ccccc1O]

An article published in Clinical Therapeutics, 1984; 6(4): 388-403 discloses treatment of arthritis with 3 grams of salsalate daily (two 750-mg tablets twice daily) for 15 days. The incidence of side effects experienced with previous therapy was reduced during salsalate administration. Patient compliance with the regimen was greater. The findings show salsalate to be effective and safe in ameliorating the symptoms of arthritic disease. The convenient twice-daily dosage regimen makes this drug particularly suitable for chronic use.

A study shows that salsalate produced a comparable clinical improvement to that with aspirin, and similar serum salicylate levels in patients with osteoarthrosis of the hip or knee. Salsalate, however, was significantly superior to aspirin with regard to side-effects and faecal occult blood loss (Current Medicinal Research and Opinion 1978; 5(6):450-3).

Salsalate has a very unpleasant taste and causes irritation of the mucous membranes of the esophagus. Known salsalate tablets overcome this problem by either film coating or by including excipients in an amount great enough to mask the taste and irritation.
For example, DISALCID™ (commercially available from Riker Laboratories, Inc., St. Paul, Minn.) is supplied as a tablet coated with hydroxypropyl methylcellulose and additionally containing magnesium stearate, microcrystalline cellulose, polyethylene glycol, polysorbate 80, starch, talc, and dye (Physician's Desk Reference, 1988, 42, 1678).

Salsalate is generally non-compressible and shows a wide variety of tableting characteristics depending on the method of manufacture. Salsalate tablets can be difficult to compress and may be subject to internal lamination, which may lead to a catastrophic tablet failure known as capping.

U.S. Pat. No. 5,225,201 discloses a salsalate tablet comprising hydroxypropyl cellulose as a binder substantially uniformly dispersed in the tablet. The tablets disclosed have good mechanical strength and exhibit a relatively low incidence of capping and does not require a discrete outer film coating to prevent esophageal irritation.

There is still a need for alternate pharmaceutical compositions of salsalate which can reduce irritation of the mucous membranes of the esophagus and the stomach after administration to the patients.

### SUMMARY OF THE INVENTION

In one general aspect, the present invention provides a modified release pharmaceutical composition comprising salsalate or a pharmaceutically acceptable salt thereof, wherein the composition releases substantially no drug within the first two hours of its administration. In one embodiment of this general aspect, the salsalate is the sole active ingredient in the composition.

In another general aspect, the present invention provides a modified release pharmaceutical composition comprising salsalate or a pharmaceutically acceptable salt thereof, wherein the composition comprises about 100 mg to about 1000 mg of salsalate and when administered in a fasted state provides an in-vivo plasma profile for salsalate with a mean of Cₘₐₓ less than about 30 μg/mL, a mean of AUC_{0-∞} less than about 60 μg*hr/mL; and a mean of Tₘₐₓ of at least about 2 hours.

In another general aspect, the present invention provides a modified release pharmaceutical composition comprising salsalate or a pharmaceutically acceptable salt thereof, wherein the composition comprises about 100 mg to about 1000 mg of salsalate and when administered in a fasted state provides an in-vivo plasma profile for salicylic acid with a mean of Cₘₐₓ less than about 55 μg/mL, a mean of AUC_{0-∞} less than about 500 μg*hr/mL; and a mean of Tₘₐₓ of at least about 4.5 hours.

In another general aspect, there is provided a modified release pharmaceutical composition comprising salsalate, wherein the salsalate is present in an amount of at least 50% by weight of the total composition. In another embodiment the salsalate is present in an amount of at least 70% by weight of the total composition.

In another general aspect, there is provided a pharmaceutical composition comprising salsalate, one or more enteric polymers and one or more pharmaceutically acceptable excipients.

Embodiments of the pharmaceutical composition may include one or more of the following features. For example, the pharmaceutically acceptable excipient may include a diluent, a disintegrant, a binder, a stabilizer, a buffering agent, a lubricant, a glidant, an antiadherent, a solubilizer, a sweetener, a flavoring agent, a solvent, and the like.

The enteric polymer may be mixed and/or granulated with salsalate or is coated over the core containing salsalate.

In another aspect, there is provided a pharmaceutical composition comprising salsalate and one or more pharmaceutically acceptable excipients, wherein the composition further comprises an additional active ingredient.

Embodiments of the pharmaceutical composition may include one or more of the following features. For example, the pharmaceutically acceptable excipient may include a diluent, a disintegrant, a binder, a stabilizer, a buffering agent, a lubricant, a glidant, an antiadherent, a solubilizer, a sweetener, a flavoring agent, a solvent, and the like.

In another general aspect, there is provided a process for preparing a modified release pharmaceutical composition of salsalate, wherein the process comprises mixing salsalate with one or more enteric polymers and one or more pharmaceutically acceptable excipients; and forming the mixture thus obtained into a pharmaceutical dosage form.

In another general aspect, there is provided a process for preparing a modified release pharmaceutical composition of salsalate, wherein the process comprises preparing a core comprising salsalate and one or more pharmaceutically acceptable excipients; and coating the core with a solution/suspension of one or more enteric polymers.

In another general aspect, there is provided a modified release pharmaceutical composition comprising salsalate, wherein the composition retains at least 80% of the potency of salsalate in the composition after storage for three months at 40° C. and 75% relative humidity.

In another general aspect, there is provided a modified release pharmaceutical composition comprising salsalate, wherein the composition exhibits an in vitro dissolution profile, when measured in a USP dissolution apparatus type I, at 150 rpm, at a temperature of 37.0±0.5° C. in 900 ml of 0.1 N HCl for first two hours followed by in pH 7.4 phosphate buffer, such that at most 10% of salsalate is released in the first two hours and at least 80% of salsalate is released within the next two hours.

In another general aspect, there is provided a method for reducing incidences of side effects associated with salsalate administration comprising administering to a subject in need thereof an effective amount of a modified release pharmaceutical composition comprising salsalate, wherein said pharmaceutical composition when administered in a fasted state provides an in-vivo plasma profile for salsalate with a mean of Cₘₐₓ less than about 30 μg/mL, a mean of AUC_{0-∞} less than about 60 μg*hr/mL; and a mean of Tₘₐₓ of at least about 2 hours.

In still another general aspect, there is provided a method for providing relief of the signs and symptoms of rheumatoid arthritis, osteoarthritis and related rheumatic disorder in a patient in need thereof, comprising administering to the patient about 100 mg to about 1000 mg of salsalate in one or more modified release oral dosage forms, wherein the administering step provides an in-vivo plasma profile for salsalate with a mean maximum plasma concentration (Cₘₐₓ) less than 30 μg/mL of salsalate, a mean AUC_{0-∞} less than about 60 μg*hr/mL and a mean Tₘₐₓ of at least 2 hours to the patient.

In another general aspect, there is provided a method for providing relief of the signs and symptoms of rheumatoid arthritis, osteoarthritis and related rheumatic disorder in a patient in need thereof, comprising administering to the patient about 100 mg to about 1000 mg of salsalate in one or more modified release oral dosage forms, wherein at steady state the composition has a fluctuation index about 10-30% lower than a fluctuation index achieved with an immediate-release composition of the salsalate.

In further general aspect, there is provided a pharmaceutical composition for modified release comprising salsalate or a pharmaceutically acceptable salt thereof, wherein the composition provides change in plasma concentration of salsalate as a function of time (dC/dT) over a defined period between 0 to 3 hours after administration that is less than about 65% of the dC/dT of the same quantity of an immediate release form of salsalate over said defined time period, wherein the dC/dT is measured in a single dose human pharmacokinetic study.

In a further general aspect, there is provided a pharmaceutical composition for modified release comprising salsalate or a pharmaceutically acceptable salt thereof, wherein the composition provides a change in plasma concentration of salicylic acid as a function of time (dC/dT) over a defined period between 0 to 4 hours after administration that is less than about 65% of the dC/dT of the same quantity of an immediate release form of salsalate over said defined time period, wherein the dC/dT is measured in a single dose human pharmacokinetic study.

In another general aspect, a modified release pharmaceutical composition of salsalate consists of granules of salsalate and one or more pharmaceutically acceptable excipients and an extragranular portion of one or more pharmaceutical excipients that is mixed with the granules and compressed into a tablet that is coated with an optional seal coating layer and then with a modified release coating.

In another general aspect, a modified release pharmaceutical composition of salsalate consists essentially of granules of salsalate and one or more pharmaceutically acceptable excipients and an extragranular portion of one or more pharmaceutical excipients that is mixed with the granules and compressed into a tablet that is coated with an optional seal coating layer and then with a modified release coating to control the release of the salsalate from the tablet.

The amount of salsalate used in the modified release composition having salsalate granules may be greater than 50%, preferably greater than 70% by weight of the total composition. The total weight of the salsalate composition may be between 900 to 1500 mg, if the amount of salsalate used in the composition is about 750 mg. In other embodiments the total weight of the 750 mg salsalate composition may be between 900 to 1400 mg, between 900 to 1300 mg, between 900 to 1200 mg or between 900 to 1100 mg. The weight is calculated on the basis of the coated dosage form. The total weight of the salsalate composition may be between 550 to 1000 mg, if the amount of salsalate used in the composition is about 500 mg. In other embodiments the total weight of the 500 mg salsalate composition may be between 550 to 900 mg, between 550 to 850 mg, between 550 to 800 mg or between 550 to 750 mg. The weight is calculated on the basis of the coated dosage form. The use of the term “about” to describe a 750 mg composition of salsalate is intended to convey that a composition labeled with 750 mg of salsalate may have slightly more or less salsalate and still be considered to be a 750 mg composition of the named active ingredient.

In another general aspect, a modified release pharmaceutical composition of salsalate consists of pellets of salsalate and one or more pharmaceutically acceptable excipients that is coated with an optional seal coating layer and then with a modified release coating. The pellets are filled into a capsule.

In another general aspect, a modified release pharmaceutical composition of salsalate consists essentially of pellets of salsalate and one or more pharmaceutical excipients that is coated with an optional seal coating layer and then with a modified release coating to control the release of the salsalate from the pellets. The pellets are filled into a capsule.

The amount of salsalate used in the modified release composition having salsalate pellets may be greater than 50%, preferably greater than 70% by weight of the total composition. The total weight of the salsalate composition may be between 900 to 1500 mg, if the amount of salsalate used in the composition is 750 mg. In other embodiments the total weight of the 750 mg salsalate composition may be between 900 to 1400 mg, between 900 to 1300 mg, between 900 to 1200 mg or between 900 to 1100 mg. The weight is calculated on the basis of the coated dosage form. The total weight of the salsalate composition may be between 550 to 1000 mg, if the amount of salsalate used in the composition is 500 mg. In other embodiments the total weight of the 500 mg salsalate composition may be between 550 to 900 mg, between 550 to 850 mg, between 550 to 800 mg or between 550 to 750 mg. The weight is calculated on the basis of the coated dosage form.

In another general aspect, a modified release pharmaceutical composition consists of salsalate as the sole active ingredient or consists essentially of salsalate as the active ingredient. The modified release pharmaceutical composition will include in addition to the salsalate one or more polymers to control or modify the release of the salsalate from the composition. The polymers may be enteric polymers. The modified release pharmaceutical composition may consist of a core that includes salsalate and a coating on the core to delay the release of the salsalate from the core. The coating may consist essentially of one or more polymers to delay the release of the salsalate from the core based on pH-dependent dissolution of the polymer. The coating may be free of zein as the polymer. The composition similarly may be free of zein. The core may be in the form of a compressed tablet, pellet, extrudate, etc.

The details of one or more embodiments of the invention are set forth in the description below. Other features, objects and advantages of the invention will be apparent from the description.

### DETAILED DESCRIPTION OF THE INVENTION

The inventors of the invention have discovered that when salsalate is formulated into a modified release pharmaceutical composition, it prevents irritation of the mucous membranes of the esophagus and the stomach.

The present invention provides a modified release pharmaceutical composition comprising salsalate or a pharmaceutically acceptable salt thereof, wherein the composition releases substantially no drug within first two hours of its administration.

The present invention provides a modified release pharmaceutical composition comprising salsalate or a pharmaceutically acceptable salt thereof, wherein the composition comprises about 100 mg to about 1000 mg of salsalate and when administered in a fasted state provides an in-vivo plasma profile for salsalate with a mean of Cₘₐₓ less than about 30 μg/mL, a mean of AUC_{0-∞} less than about 60 μg*hr/mL; and a mean of Tₘₐₓ at least about 2 hours.

In some embodiments, the present invention provides a modified release pharmaceutical composition comprising salsalate or a pharmaceutically acceptable salt thereof, wherein the composition comprises about 250 mg to about 750 mg of salsalate and when administered in a fasted state provides an in-vivo plasma profile for salsalate with a mean of Cₘₐₓ less than about 25 μg/mL, a mean of AUC_{0-∞} less than about 50 μg*hr/mL; and a mean of Tₘₐₓ at least about 3 hours.

In some embodiments, the present invention provides a modified release pharmaceutical composition comprising salsalate or a pharmaceutically acceptable salt thereof, wherein the composition comprises about 500 mg to about 750 mg of salsalate and when administered in a fasted state provides an in-vivo plasma profile for salsalate with a mean of Cₘₐₓ less than about 20 μg/mL, a mean of AUC_{0-∞} less than about 45 μg*hr/mL; and a mean of Tₘₐₓ at least about 4 hours.

The present invention provides a modified release pharmaceutical composition comprising salsalate or a pharmaceutically acceptable salt thereof, wherein the composition comprises about 100 mg to about 1000 mg of salsalate and when administered in a fasted state provides an in-vivo plasma profile for salicylic acid with a mean of Cₘₐₓ less than about 55 μg/mL, a mean of AUC_{0-∞} less than about 500 μg*hr/mL; and a mean of Tₘₐₓ at least about 4.5 hours.

In some embodiments, the present invention provides a modified release pharmaceutical composition comprising salsalate or a pharmaceutically acceptable salt thereof, wherein the composition comprises about 250 mg to about 750 mg of salsalate and when administered in a fasted state provides an in-vivo plasma profile for salicylic acid with a mean of Cₘₐₓ less than about 40 μg/mL, a mean of AUC_{0-∞} less than about 475 μg*hr/mL; and a mean of Tₘₐₓ at least about 6 hours.

In some embodiments, the present invention provides a modified release pharmaceutical composition comprising salsalate or a pharmaceutically acceptable salt thereof, wherein the composition comprises about 500 mg to about 750 mg of salsalate and when administered in a fasted state provides an in-vivo plasma profile for salicylic acid with a mean of Cₘₐₓ less than about 35 μg/mL, a mean of AUC_{0-∞} less than about 450 μg*hr/mL; and a mean of Tₘₐₓ at least about 7 hours.

The term “salsalate” used throughout the specification refers to not only salsalate per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof. The amount of salsalate used in the present invention is in the range less than or equal to 3000 mg/day in a single or divided doses. The amount of salsalate used in the composition may be greater than 50%, preferably greater than 70% by weight of the total composition.

The total weight of the salsalate composition may be between 900 to 1500 mg, if the amount of salsalate used in the composition is 750 mg. In other embodiments the total weight of the 750 mg salsalate composition may be between 900 to 1400 mg, between 900 to 1300 mg, between 900 to 1200 mg or between 900 to 1100 mg. The weight is calculated on basis of the coated dosage form.

The total weight of the salsalate composition may be between 550 to 1000 mg, if the amount of salsalate used in the composition is 500 mg. In other embodiments the total weight of the 500 mg salsalate composition may be between 550 to 900 mg, between 550 to 850 mg, between 550 to 800 mg or between 550 to 750 mg. The weight is calculated on basis of the coated dosage form.

The term “modified release” used herein may be understood to include extended release, controlled release, sustained release, delayed release and pulsatile release. A “delayed release” composition may be designed to delay the release of the drug for a specified period. Delayed release pharmaceutical compositions of the present invention include those that exhibit a delayed-release, e.g., compositions that only begin releasing the drug after a fixed period of time. The delayed release pharmaceutical compositions of the present invention may include the compositions which may release substantially no drug within first two hours and after completion of that the composition may release more than 80% of the drug within next two hours. The composition may release less than about 30%, preferably less than 20%, more preferably less than 10% of total drug within one hour after administration. In some embodiments, the delayed release composition includes an enteric coating, which is a barrier applied to oral drug that prevents release of the drug before it reaches the small intestine. Delayed release formulations, such as enteric coatings, prevent drugs having an irritant effect on the stomach, such as salsalate, from dissolving in the stomach. Such coatings are also used to protect acid-unstable drugs from the stomach's acidic exposure, delivering them instead to a basic pH environment (intestine's pH 5.5 and above) where they do not degrade, and give their desired action.

The delayed release property of a dosage form may be achieved by using one or more enteric polymers. “Enteric polymer” used in the invention may be selected from hydroxypropyl methylcellulose phthalate, cellulose acetate phthalate, cellulose acetate succinate, methylcellulose phthalate, hydroxypropyl methylcellulose phthalate, ethylhydroxycellulose phthalate, polyvinyl acetate phthalate, polyvinyl butyrate acetate, vinyl acetate-maleic anhydride copolymer, styrene-maleic mono-ester copolymer, carboxymethyl ethylcellulose, methyl methacrylate-methacrylic acid copolymer (Eudragit L-100 (methacrylic acid copolymer L) or Eudragit S-100 (methacrylic acid copolymer S)), methacrylic acid-ethyl acrylate copolymer (Eudragit L100-55 (dried methacrylic acid copolymer LD) or Eudragit L30D-55 (methacrylic acid copolymer LD)), methacrylic acid-methyl acrylate-methyl methacrylate copolymer (Eudragit FS30D), hydroxypropyl cellulose acetate succinate (HPMCAS) and shellac.

In some embodiments, the enteric polymer may be mixed and/or granulated with the drug to prepare final composition. Alternatively, the solution or suspension of one or more enteric polymers may be coated on the core containing the drug. The core may be prepared as per the knowledge of the skilled artisan. The core may be a mixture of drug and excipients or it may be an inert core, coated with a drug layer. There might be an intermediate layer between the drug core and the enteric layer.

The delayed release property of the dosage form, where present, may be achieved by using press-coating over drug-containing core. The press-coat may comprise hydrophilic or hydrophobic rate controlling materials.

In specific embodiments, the modified release pharmaceutical composition may further comprise a sustained release component or a controlled release component in a single dose formulation. The sustained release or controlled release component may comprise hydrophilic or hydrophobic rate controlling materials.

Suitable hydrophilic rate controlling materials are selected from, but are not limited to alkyl celluloses such as methyl cellulose; hydroxyalkyl celluloses, for example, hydroxymethyl cellulose, hydroxyethyl cellulose, hydroxypropyl cellulose, and hydroxybutyl cellulose; hydroxyalkyl alkyl celluloses such as hydroxyethyl methyl cellulose and hydroxypropyl methyl cellulose; carboxyalkyl cellulose esters; crosslinked cellulose derivatives such as crosslinked sodium carboxymethyl cellulose; crosslinked polyvinyl pyrrolidone and vinyl acetate (commercially available grade such as Kollidon VA64); polysaccharides such as galactomannans, tragacanth, agar, guar gum, and polyfructans; polyvinyl alcohol; polyethylene glycol, polyvinylpyrrolidone, copolymers of polyvinylpyrrolidone with vinyl acetate; combinations of polyvinyl alcohol and polyvinylpyrrolidone; and polyalkylene oxides such as polyethylene oxide and polypropylene oxide and copolymers of ethylene oxide and propylene oxide.

Suitable hydrophobic rate controlling materials for coating are selected from, but are not limited to one or more of glyceride (e.g., glyceryl behenate, glyceryl trimyristate, glyceryl trilaurate, glyceryl tristearate, glyceryl monostearate, glyceryl palmitostearate, or glyceryl triacetate), stearic acid, hydrogenated castor oil, a hydrogenated vegetable oil, a water insoluble cellulose (e.g., ethyl cellulose, cellulose acetate, cellulose acylate, cellulose diacylate, cellulose triacylate, cellulose acetate butyrate, cellulose acetate propionate, nitrocellulose, cellulose diacetate, or cellulose triacetate), a wax or a wax-like substance (e.g., carnauba wax, cetyl esters wax, beeswax, castor wax, cationic emulsifying wax, cetrimide emulsifying wax, an emulsifying wax, microcrystalline wax, a nonionic wax, a nonionic emulsifying wax, paraffin, petroleum wax, petroleum ceresin wax, spermaceti wax, white wax, or yellow wax), a fat, an oil, a fatty acid, an emulsifier, a modified starch, a fatty alcohol, a protein (e.g., zein), shellac, or a polymer (e.g., a polyolefin, a polyurethane, a polyvinylchloride, a polyvinyl acetate, an acrylic acid polymer, a methacrylic acid polymer); cetostearyl alcohol, stearyl alcohol, and the like.

The coating composition may optionally include other excipients, such as binders, lubricants, processing aids, pH buffers, glidants, colorants, and the like, which can be the same or different as those in the core composition, if any.

The pharmaceutical compositions as described herein may be prepared by processes known to the person having ordinary skill in the art of pharmaceutical technology such as direct compression, wet granulation, dry granulation or melt granulation.

Suitable final dosage form may comprise one or more of tablets, multilayered tablets, capsules, pellets, granules, spheroids, beads, minitablets in a capsule, pellets in a capsule, granules in a capsule, and powder. Further, the powder or granules can be suspended to give a pharmaceutically acceptable oral suspension.

The pharmaceutically acceptable excipients may include one or more of diluents, disintegrants, binders, stabilizers, buffering agents, lubricants, glidants, antiadherents, solubilizers, taste masking agents, sweeteners, flavoring agents and solvents.

Suitable diluents may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol or erythritol; and mixtures thereof. The diluent may be added to increase the bulk volume of the powder to facilitate granulation or compression.

Suitable disintegrants may include one or more of croscarmellose sodium, crospovidone, sodium starch glycolate, corn starch, potato starch, maize starch and modified starches, calcium silicates, and low substituted hydroxypropylcellulose. The amount of disintegrating agent is preferably in the range of 5% to 35% w/w of the composition.

Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins, and the like.

Suitable stabilizers may include, especially in the sprinkle oral formulation, alkali-metals and alkaline earth metals, bases of phosphates and organic acid salts and organic amines or mixtures thereof. Stabilizers may be selected from sodium citrate, NaCl, K₂HPO₄, meglumine, sodium ascorbate, KCl, sodium sulfite, Poloxamer 188/407, polyethylene glycol, glyceryl monooleate, alginic acid, albumin, ammonium alginate, ascorbic acid, ascorbyl palmitate, bentonite, butylated hydroxytoluene, calcium alginate, calcium state, carboxymethylcellulose calcium, carboxymethylcellulose sodium, carrageenan, ceratonia, colloidal silicon dioxide, cyclodextrins, diethanolamine, edetates, ethylene glycol palmisterate, glycerin monosterate, guargum, magnesium aluminium silicate, lecithin, hypromellose, hydroxypropyl cellulose, polacrilin potassium, pectin, poloxamer, polyvinyl alcohol, propyl gallate, propylene glycol, xylitol, zinc acetate, raffinose, sodium borate, trehalose, propylene glycol alginate, sulfobutylether beta-cyclo dextrin, or mixtures thereof or the well-known stabilizers known to a person skilled in the art.

Suitable buffering agents may include one or more of ammonia solution, calcium carbonate, calcium phosphate, citric acid, sodium phosphate, diethanol amine, malic acid, monosodium glutamate, phosphoric acid, potassium citrate, sodium acetate, sodium bicarbonate, sodium borate, sodium citrate, sodium hydroxide, sodium lactate, triethanol amine, or mixtures thereof or the well-known buffering agents known to a person skilled in the art.

Suitable lubricants, glidants or anti-adherent agents may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and mixtures thereof. It would be appreciated that a person skilled in the art is cognizant of the fact that lubricant, glidant or anti-tacking agent may be used interchangeably. The lubricant, glidant or anti-tacking agent may be present in an amount ranging from 0.1% to 10% w/w of the composition.

Suitable solubilizers may include one or more of sodium lauryl sulphate, polyvinyl pyrrolidone, lactose, mannitol, cyclodextrine or polyethylene glycols.

Suitable surfactants may include one or more of anionic, cationic, non-ionic or amphoteric surfactants or those known to the person skilled in the art. Non-limiting examples of surfactants include polyoxyethylene-polyoxypropylene co-polymers and block co-polymers, commercially available as Pluronic™ or Poloxamer™, ethoxylated cholesterins, commercially available as Solulan™ vitamin derivatives, e.g. vitamin E derivatives such as tocopherol polyethylene glycol succinate (TPGS), sodium dodecylsulfate or sodium lauryl sulfate; a bile acid or salt thereof, for example cholic acid, glycolic acid, or a salt.

Suitable taste masking agents may include one or more of polymers, sweeteners and flavors. Most preferred polymers may include one or more of cellulose acetate, polymethacrylates, hydroxypropylmethylcellulose, hydroxypropylcellulose, or hydroxylethylcellulose.

Suitable sweeteners may include one or more of saccharides such as sucrose, dextrose, glucose, maltose, dextrins, D-tagatose, trehalose, dried invert sugar, fructose, levulose, galactose, corn syrup solids, and the like, alone or in combination. Other examples of sweeteners include sodium saccharin; aspartame; sugarless sweeteners including polyhydric alcohols such as sorbitol, mannitol, xylitol, glycerol, hydrogenated starch hydrolysates, maltitol, isomaltitol, erythritol, lactitol, and the like, alone or in combination.

Suitable flavoring agents may include one or more of cinnamon, wintergreen, eucalyptus, spearmint, peppermint, menthol, anise as well as fruit flavors such as apple, pear, peach, strawberry, cherry, apricot, orange, watermelon, banana and the like; bean-derived flavors, such as coffee, cocoa, and the like, or mixtures thereof.

In some of the embodiments, the pharmaceutical composition of the invention may further comprise another active ingredient, preferably selected from the proton pump inhibitors. Generally, proton pump inhibitors, their single enantiomers or alkaline salts thereof, are used for the prevention and treatment of gastric acid related diseases including, but not limited to, reflux esophagitis, gastritis, duodenitis, gastric ulcer and duodenal ulcer. Additionally, these proton pump inhibitors may be used for the treatment of other gastrointestinal disorders where gastric acid inhibitory effect is desirable, such as patients with Non Ulcer Dyspepsia, in patients with symptomatic gastro-esophageal reflux disease, in patients with gastrinomas, and in particular in patients on NSAID therapy. The term “proton pump inhibitors” or “acid sensitive/unstable proton pump inhibitors” or “PPIs” used throughout the specification refers to agents which inhibit gastric acid secretion by inhibiting H+/K+ ATPase, the enzyme involved in the final step of hydrogen ion production in the parietal cells. The term “proton pump inhibitor” includes, but is not limited to benzimidazole compounds such as omeprazole, lansoprazole, rabeprazole, pantoprazole and leminoprazole, including isomers, enantiomers and tautomers thereof, and alkaline salts thereof (such as magnesium, sodium).

In one embodiment, a pharmaceutical composition may be prepared by mixing and/or granulating salsalate with one or more enteric polymers and one or more pharmaceutically acceptable excipients; compressing the mixture or granules to form a tablet; and optionally coating the tablet.

In another embodiment, a pharmaceutical composition may be prepared by preparing a core comprising salsalate and one or more pharmaceutically acceptable excipients; optionally coating the core with an intermediate layer; and coating with a layer comprising one or more enteric polymers.

In another embodiment, a pharmaceutical composition may be prepared by preparing an inert core; coating the inert core with a solution/suspension comprising salsalate and one or more pharmaceutically acceptable excipients; coating with one or more enteric layers; and optionally coating with a functional/non-functional layer. The non-functional layer may also comprise of flavors like vanilla, cinnamon, wintergreen, eucalyptus, spearmint, peppermint, menthol, anise as well as fruit flavors such as apple, pear, peach, strawberry, cherry, apricot, orange, watermelon, banana and the like; bean-derived flavors, such as coffee, cocoa, and the like, or mixtures thereof.

In still another embodiment, a pharmaceutical composition may be prepared by mixing and/or granulating salsalate with one or more pharmaceutically acceptable excipients; filling the mixture or granules into a capsule; and coating the capsule with an enteric coating.

The pharmaceutical composition according to the invention may retain at least 80% of the potency of salsalate in the composition after storage for three months at 40° C. and 75% relative humidity.

The pharmaceutical composition according to the invention exhibits an in vitro dissolution profile, when measured in a USP dissolution apparatus type I, at 150 rpm, at a temperature of 37.0±0.5° C. in 900 ml of 0.1 N HCl, such that at most 20%, preferably at most 10%, most preferably at most 5% of salsalate is released in first one hour, preferably in first two hours. In most preferred embodiment of the invention, the composition of the present invention releases substantially no drug within first two hours after administration.

The modified release composition demonstrates good tolerability. Cₘₐₓ (maximum plasma concentration) is less than about 85% of the immediate release tablets when administered as a single dose. AUC (area under the curve, a measure of bioavailability) is within 75% to 130% of the immediate release tablets administered as a single dose. This range is considered equivalent with respect to overall systemic exposure.
Prolonging the time to maximum plasma concentration (Tₘₐₓ) as compared to immediate release tablet, is related to the release rate of the drug in the use environment. The release rate of the drug depends on many factors, including the composition of the solid dosage forms and the dissolution properties.

The control of drug release is particularly desirable for reducing and delaying the peak plasma level while maintaining the extent of drug bioavailability. The therapeutic levels are therefore achieved while minimizing debilitating side-effects that are usually associated with immediate release formulations. Furthermore, as a result of delay in the time to obtain peak serum or plasma level and the extended period of time at the therapeutically effective serum or plasma level, the dosage frequency is reduced to, for example, once or twice daily dosage, thereby improving patient compliance and adherence. For example, the side effects including cardiovascular side effects and gastrointestinal side effects may be lessened in severity and frequency through the use of modified release methods that shift the Tₘₐₓ to longer times, thereby reducing the dC/dT of the drug. Reducing the dC/dT of the drug not only increases Tₘₐₓ, but also reduces the drug concentration at Tₘₐₓ and reduces the Cₘₐₓ/Cₘₑₐₙ ratio providing a more constant amount of drug to the subject being treated over a given period of time, enabling increased dosages for appropriate indications.

As used herein, “C” refers to the concentration of an active pharmaceutical ingredient in a biological sample, such as a patient sample (e.g. blood, serum, and cerebrospinal fluid). The time required to reach the maximal concentration (“Cₘₐₓ”) in a particular patient sample type is referred to as the “Tₘₐₓ”. The change in concentration is termed “dC” and the change over a prescribed time is “dC/dT”.

The term “substantially constant” with respect to the serum level of active moiety or moieties means that the serum profile after administration of the controlled release formulation does essentially not exhibit any substantial peak values. This may also be expressed mathematically by reference to the “fluctuation index” (FI) for the serum concentration of (unbound) active moiety (or sum of active moieties when relevant), where the fluctuation index FI is calculated as:

FI=(Cₘₐₓ−Cₘᵢₙ)/AUCτ/τ

wherein Cₘₐₓ and Cₘᵢₙ are the maximum and minimum concentrations, respectively, of the active moiety, AUCτ is the area under the serum concentration profile (concentration vs. time curve), and τ is the length of the dosage interval during the time τ. The modified release composition according to the present invention at steady state has a fluctuation index about 10-30% lower than a fluctuation index achieved with an immediate-release composition of the salsalate.

In other embodiment, there is provided a method of treating signs and symptoms of rheumatoid arthritis, osteoarthritis and related rheumatic disorders which comprises administering to a human patient in need thereof the modified release pharmaceutical composition of salsalate as per the invention.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

### Modified Release Tablets of Salsalate (750 Mg)

| | | | |
|---|---|---|---|
| Sr. | | Quantity | |
| No. | Ingredients | mg/Tab | % w/w |

Intragranular

| | | | |
|---|---|---|---|
| 1 | Salsalate | 750.00 | 75.76 |
| 2 | Microcrystalline cellulose | 64.50 | 6.52 |
| 3 | Croscarmellose sodium | 18.00 | 1.82 |

Granulation

| | | | |
|---|---|---|---|
| 4 | Hypromellose 2910 5 cps | 36.00 | 3.64 |
| 5 | Purified water | q.s. | — |

Extragranular

| | | | |
|---|---|---|---|
| 6 | Colloidal silicon dioxide | 4.50 | 0.45 |
| 7 | Croscarmellose sodium | 18.00 | 1.82 |

Lubrication

| | | | |
|---|---|---|---|
| 8 | Stearic acid | 9.00 | 0.91 |

Seal coating

| | | | |
|---|---|---|---|
| 9 | Hypromellose 2910 5 cps | 14.25 | 1.44 |
| 10 | Polyethylene glycol 8000 | 0.75 | 0.08 |
| 11 | Purified water | q.s. | — |

Functional coating

| | | | |
|---|---|---|---|
| 12 | Acrylic acid copolymer | 67.26 | 6.79 |
| 13 | Polyethylene glycol 8000 (Powder) | 7.50 | 0.76 |
| 14 | D & C Yellow # 10 AL Lake | 0.24 | 0.02 |
| 15 | Purified water | q.s. | — |

| | | |
|---|---|---|
| Total | 990.00 | 100.00 |

### Process:

Salsalate, microcrystalline cellulose and croscarmellose sodium were mixed and granulated with a dispersion of hypromellose in purified water. The granules were dried and mixed with croscarmellose sodium and colloidal silicon dioxide. The granules were lubricated with stearic acid. The lubricated mixture was compressed to provide tablets. The tablets were seal coated with a dispersion of hypromellose in purified water. The coated tablets were again coated with a dispersion of acrylic acid copolymer.

### Dissolution Profile for Tablets of Example 1

| | | | Salsalate |
|---|---|---|---|
| Sr. | | Time | DR Tablet |
| No. | Phase | (Min) | 750 mg |
| 1 | Acid Phase | 120 | 0 |
| 2 | Buffer Phase | 5 | 18 |
| 3 | | 10 | 97 |
| 4 | | 15 | 102 |
| 5 | | 20 | 102 |
| 6 | | 30 | 103 |
| 7 | | 45 | 102 |
| 8 | | 60 | 103 |

Acid Phase: 0.1N HCl/900 ml/Apparatus I (Basket)/150 RPM
Buffer Phase: 0.25M pH 7.4 Phosphate Buffer/900 mL/Apparatus I (Basket)/150 RPM

### Stability Data for Tablets of Example 1

| | | Stability Condition | | | |
|---|---|---|---|---|---|
| | | | | | 25° C., |
| | | 40° C., 75% RH | | | 60% RH |
| Tests | Initial | 1M | 2M | 3M | 3 M |

Related Compounds

| | | | | | |
|---|---|---|---|---|---|
| Salicylic acid | 0.43% | 0.8% | 1.0% | 1.3% | 0.7% |
| Trisalicylic acid | 0.29% | 0.3% | 0.3% | 0.3% | 0.3% |
| Single Maximum | 0.07% | 0.01% | 0.00% | 0.01% | 0.02% |
| Unknown Impurity | | | | | |
| Total Impurities | 0.08% | 0.01% | 0.01% | 0.01% | 0.02% |
| (Excluding Salicylic Acid | | | | | |
| & Trisalicylic Acid) | | | | | |

### Example 2

### Modified Release Tablets of Salsalate (750 Mg)

| | | | |
|---|---|---|---|
| Sr. | | Quantity | |
| No. | Ingredients | mg/Tab | % w/w |

Intragranular

| | | | |
|---|---|---|---|
| 1 | Salsalate | 750.00 | 75.00 |
| 2 | Microcrystalline cellulose | 55.50 | 5.55 |
| 3 | Croscarmellose sodium | 27.00 | 2.70 |

Granulation

| | | | |
|---|---|---|---|
| 4 | Hypromellose 2910 5 cps | 36.00 | 3.60 |
| 5 | Purified water | q.s. | — |

Extragranular

| | | | |
|---|---|---|---|
| 6 | Colloidal silicon dioxide | 4.50 | 0.45 |
| 7 | Croscarmellose sodium | 18.00 | 1.80 |

Lubrication

| | | | |
|---|---|---|---|
| 8 | Stearic acid | 9.00 | 0.90 |

Seal coating

| | | | |
|---|---|---|---|
| 9 | Hypromellose 2910 5 cps | 14.25 | 1.43 |
| 10 | Polyethylene glycol 8000 | 0.75 | 0.08 |
| 11 | Ethanol | q.s. | — |
| 12 | Purified water | q.s. | — |

Functional coating

| | | | |
|---|---|---|---|
| 13 | Hydroxypropyl methylcellulose | 76.50 | 7.65 |
| | phthalate (HPMCP) | | |
| 14 | Polyethylene glycol 8000 (Powder) | 8.50 | 0.85 |
| 15 | Ethanol | q.s. | — |
| 16 | Purified water | q.s. | — |

| | | |
|---|---|---|
| Total | 1000.00 | 100.00 |

### Process:

Salsalate, microcrystalline cellulose and croscarmellose sodium were mixed and granulated with a dispersion of hypromellose in purified water. The granules were dried and mixed with croscarmellose sodium and colloidal silicon dioxide. The granules were lubricated with stearic acid. The lubricated mixture was compressed to provide tablets. The tablets were seal coated with a dispersion of hypromellose in purified water. The coated tablets were again coated with a dispersion of HPMC phthalate.

### Dissolution Profile for Tablets of Example 2

| | | | Salsalate |
|---|---|---|---|
| Sr. | | Time | DR Tablet |
| No. | Phase | (Min) | 750 mg |
| 1 | Acid Phase | 120 | 0 |
| 2 | Buffer Phase | 5 | 52 |
| 3 | | 10 | 93 |
| 4 | | 15 | 97 |
| 5 | | 20 | 98 |
| 6 | | 30 | 98 |
| 7 | | 45 | 97 |
| 8 | | 60 | 98 |

Acid Phase: 0.1N HCl/900 ml/Apparatus I (Basket)/150 RPM
Buffer Phase: 0.25M pH 7.4 Phosphate Buffer/900 mL/Apparatus I (Basket)/150 RPM

### Stability Data for Tablets of Example 2

| | | Stability Condition | | |
|---|---|---|---|---|
| | | 40° C., | | |
| | | 75% RH | 25° C., 60% RH | |
| Tests | Initial | 3 M | 3 M | 6 M |

Related Compounds

| | | | | |
|---|---|---|---|---|
| Salicylic acid | 0.2% | 1.5% | 0.5% | 0.7% |
| Trisalicylic acid | 0.2% | 0.4% | 0.3% | 0.3% |
| Single Maximum | 0.01% | 0.06% | 0.05% | 0.07% |
| Unknown Impurity | | | | |
| Total Impurities | 0.0% | 0.1% | 0.1% | 0.1% |
| (Excluding Salicylic Acid | | | | |
| & Trisalicylic Acid) | | | | |

The tablets of salsalate as per Example 2 were administered to healthy human volunteers in the form of tablets that contain 750 mg of salsalate each. These modified release compositions were administered to humans after a fasting period of at least about 8 hours. The blood samples were collected from each volunteer before administration and at several time points after administration (e.g., at 30 minutes, 1 hour, 2 hours, 3 hours, 4 hours, 5 hours, 6 hours, 7 hours, 8 hours, 10 hours, 12 hours). The plasma samples from each volunteer were assayed for salsalate and salicylic acid quantification using a validated method.

The tablets of Example 2 provided pharmacokinetic (PK) parameters substantially as shown in the below mentioned Table. These PK parameters are expressed as geometric mean (geometric CV %).

| | Mean Cₘₐₓ | Mean AUC_{0-∞} | Mean Tₘₐₓ |
|---|---|---|---|
| | (μg/mL) | (μg * hr/mL) | (hour) |
| Salsalate | 19.24 (±10.26) | 46.04 (±15.26) | 4.32 (±1.95) |
| Salicylic acid | 38.77 (±17.47) | 333.36 (±178.11) | 6.04 (±1.75) |

### Example 3

### Modified Release Capsules of Salsalate (750 Mg)

| Sr. | | Quantity | |
|---|---|---|---|
| No. | Ingredients | mg/Tab | % w/w |

Intragranular

| | | | |
|---|---|---|---|
| 1 | Salsalate | 750.00 | 72.82 |
| 2 | Microcrystalline Cellulose (Comprecel 101D+) | 55.50 | 5.39 |
| 3 | Croscarmellose Sodium (Ac-Di-Sol) | 27.00 | 2.62 |

Granulation

| | | | |
|---|---|---|---|
| 4 | Hypromellose 2910 5 cps (Methocel E5 LV) | 36.00 | 3.50 |
| 5 | Purified Water^{#} | q.s. | — |

Extragranular

| | | | |
|---|---|---|---|
| 6 | Colloidal Silicon Dioxide (Aerosil-200 Pharma) | 4.50 | 0.44 |
| 7 | Croscarmellose Sodium (Ac-Di-Sol) | 18.00 | 1.75 |

Lubrication

| | | | |
|---|---|---|---|
| 8 | Stearic Acid (Stellipress Micro) | 9.00 | 0.87 |

Seal Coating

| | | | |
|---|---|---|---|
| 9 | Hypromellose 2910 5 cps (Methocel E5 LV) | 14.25 | 1.38 |
| 10 | Polyethylene Glycol 8000 (Powder) (Carbowax | 0.75 | 0.07 |
| | Sentry) | | |
| 11 | Ethanol^{#} | q.s. | |
| 12 | Purified Water^{#} | q.s. | — |

Functional Coating

| | | | |
|---|---|---|---|
| 13 | Hypromellose Phthalate (HPMC HP 50) | 76.50 | 7.43 |
| 14 | Polyethylene Glycol 8000 (Powder) (Carbowax | 8.50 | 0.83 |
| | Sentry | | |
| 15 | Ethanol^{#} | q.s. | — |
| 16 | Purified Water^{#} | q.s. | — |

Flavor Coating

| | | | |
|---|---|---|---|
| 17 | Opadry White 33F28398 | 27.00 | 2.62 |
| 18 | Vanilla Flavor | 3.00 | 0.29 |
| 19 | Purified Water^{#} | q.s. | — |
| | Total | 1000.00 | 100.01 |
| | | | ≈100.00 |

^{#}Does not remain in product except in traces.

### Process:

Salsalate, microcrystalline cellulose and croscarmellose sodium were mixed and granulated with a dispersion of hypromellose in purified water. The granules were dried and mixed with croscarmellose sodium and colloidal silicon dioxide. The granules were lubricated with stearic acid. The lubricated mixture was compressed to provide tablets. The tablets were seal coated with a dispersion of hypromellose and polyethylene glycol in purified water and ethanol. The coated tablets were then functionally coated with a dispersion of HPMC phthalate and polyethylene glycol in purified water and ethanol.
The coated tablets were then coated with a flavor coating of Opadry white and vanilla flavor in purified water.

### Dissolution Profile for Tablets of Example 3

| Sr. | | Time | Salsalate DR Tablet |
|---|---|---|---|
| No. | Phase | (Min) | 750 mg |
| 1 | Acid Phase | 120 | 0 |
| 2 | Buffer | 5 | 54 |
| 3 | Phase | 10 | 92 |
| 4 | | 15 | 96 |
| 5 | | 20 | 96 |
| 6 | | 30 | 96 |
| 7 | | 45 | 97 |
| 8 | | 60 | 97 |

Acid Phase: 0.1N HCl/900 ml/Apparatus I (Basket)/150 RPM
Buffer Phase: 0.25M pH 7.4 Phosphate Buffer/900 mL/Apparatus I(Basket)/150 RPM

### Stability Data for Tablets of Example 3

| Tests | Initial |
|---|---|
| Salicylic acid | 0.2% |
| Trisalicylic acid | 0.2% |
| Single Maximum | 0.01% |
| Unknown Impurity | |
| Total Impurities | 0.0% |
| (Excluding Salicylic Acid | |
| & Trisalicylic Acid) | |

### Example 4

### Modified Release Capsules of Salsalate (500 Mg)

| | |
|---|---|
| Sr. | Quantity |

| | | | |
|---|---|---|---|
| No. | Ingredients | mg/Cap | % w/w |

Intragranular

| | | | |
|---|---|---|---|
| 1 | Salsalate | 500.00 | 71.43 |
| 2 | Microcrystalline cellulose | 58.00 | 8.29 |
| 3 | Croscarmellose sodium | 36.00 | 5.14 |

Granulation

| | | | |
|---|---|---|---|
| 4 | Hypromellose 2910 5 cps | 24.00 | 3.43 |
| 5 | Glycerin | 2.00 | 0.29 |
| 6 | Purified water | q.s. | — |

Seal Coating

| | | | |
|---|---|---|---|
| 7 | Hypromellose 2910 5 cps | 14.25 | 2.04 |
| 8 | Polyethylene glycol 8000 | 0.75 | 0.11 |
| 9 | Purified water | q.s. | — |

Functional coating

| | | | |
|---|---|---|---|
| 10 | Acrylic acid copolymer | 58.50 | 8.36 |
| 11 | Polyethylene glycol 8000 | 6.50 | 0.93 |
| 12 | Purified water | q.s. | — |

Capsule filling

| | | | |
|---|---|---|---|
| 13 | Size ‘00’el Hard Gelatin Capsule | 1 # | — |

| | | |
|---|---|---|
| Total | 700.00 | 100.00 |

### Process:

Salsalate, microcrystalline cellulose and croscarmellose sodium were mixed and granulated with a dispersion of hypromellose and glycerin in purified water. The wet granules were extruded and spheronized to provide wet pellets. These pellets were dried and were seal coated with a dispersion of hypromellose in purified water. The coated pellets were again coated with a dispersion of acrylic acid copolymer. The final coated pellets were sized and filled into the capsules.

### Dissolution Profile for Capsules of Example 4

| Sr. | | Time | Salsalate Modified-Release |
|---|---|---|---|
| No. | Phase | (Min) | Capsules 500 mg |
| 1 | Acid | 120 | 1 |
| | Phase | | |
| 2 | Buffer | 5 | 55 |
| 3 | Phase | 10 | 77 |
| 4 | | 15 | 87 |
| 5 | | 20 | 93 |
| 6 | | 30 | 94 |
| 7 | | 45 | 97 |
| 8 | | 60 | 94 |

Acid Phase: 0.1N HCl/900 ml/Apparatus I (Basket)/150 RPM
Buffer Phase: 0.25M pH 7.4 Phosphate Buffer/900 mL/Apparatus I (Basket)/150 RPM

### Stability Data for Capsules of Example 4

| | | Stability Condition | | | |
|---|---|---|---|---|---|
| | | | | | 25° C., |
| | | | | | 60% |
| | | 40° C., 75% RH | | | RH |
| Tests | Initial | 1 M | 2 M | 3 M | 3 M |

Related Compounds

| | | | | | |
|---|---|---|---|---|---|
| Salicylic acid | 0.6% | 1.52% | 1.89% | 2.13% | 0.47% |
| Trisalicylic acid | 0.3% | 0.42% | 0.49% | 0.45% | 0.30% |
| Single Maximum | 0.01% | 0.04% | 0.04% | 0.05% | 0.02% |
| Unknown Impurity | | | | | |
| Total Impurities | 0.01% | 0.05% | 0.06% | 0.08% | 0.04% |
| (Excluding Salicylic | | | | | |
| Acid & Trisalicylic Acid) | | | | | |

### Example 5

### Modified Release Tablets of Salsalate (500 Mg)

| Sr. | | Quantity | |
|---|---|---|---|
| No. | Ingredients | mg/Tab | % w/w |

Intragranular

| | | | |
|---|---|---|---|
| 1 | Salsalate | 500.00 | 72.82 |
| 2 | Microcrystalline cellulose | 37.00 | 5.39 |
| 3 | Croscarmellose sodium | 18.00 | 2.62 |

Granulation

| | | | |
|---|---|---|---|
| 4 | Hypromellose 2910 5 cps | 24.00 | 3.50 |
| 5 | Purified water | q.s. | — |

Extragranular

| | | | |
|---|---|---|---|
| 6 | Colloidal silicon dioxide | 3.00 | 0.44 |
| 7 | Croscarmellose sodium | 12.00 | 1.75 |

Lubrication

| | | | |
|---|---|---|---|
| 8 | Stearic acid | 6.00 | 0.87 |

Seal coating

| | | | |
|---|---|---|---|
| 9 | Hypromellose 2910 5 cps | 9.50 | 1.38 |
| 10 | Polyethylene glycol 8000 | 0.50 | 0.07 |
| 11 | Ethanol | q.s. | — |
| 12 | Purified water | q.s. | — |

Functional coating

| | | | |
|---|---|---|---|
| 13 | Hydroxypropyl methylcellulose | 51.00 | 7.43 |
| | phthalate (HPMCP) | | |
| 14 | Polyethylene glycol 8000 (Powder) | 5.67 | 0.83 |
| 15 | Ethanol | q.s. | — |
| 16 | Purified water | q.s. | — |

Flavor coat

| | | | |
|---|---|---|---|
| 17 | Opadry | 18.00 | 2.62 |
| 18 | Vanilla flavor | 2.00 | 0.29 |
| 19 | Purified water | q.s. | — |

| | | |
|---|---|---|
| Total | 686.67 | 100.00 |

### Process:

Salsalate, microcrystalline cellulose and croscarmellose sodium were mixed and granulated with a dispersion of hypromellose in purified water. The granules were dried and mixed with croscarmellose sodium and colloidal silicon dioxide. The granules were lubricated with stearic acid. The lubricated mixture was compressed to provide tablets. The tablets were seal coated with a dispersion of hypromellose in purified water. The coated tablets were again coated with a dispersion of HPMCP. The tablets were then coated with a flavor coat containing vanilla flavor.

### Example 6

| | | |
|---|---|---|
| | | Quantity |
| Sr. No. | Ingredients | (% w/w) |

MR1 Component:

| | | |
|---|---|---|
| 1 | Salsalate | 20-40 |
| 2 | Microcrystalline cellulose | 4-10 |
| 3 | Croscarmellose sodium | 0.5-10 |
| 4 | Hypromellose | 0.5-10 |
| 5 | Purified water | q.s. |

MR2 Component:

| | | |
|---|---|---|
| 1 | Salsalate | 30-50 |
| 2 | Microcrystalline cellulose | 5-20 |
| 3 | Croscarmellose sodium | 0.5-10 |
| 4 | Hypromellose | 0.1-10 |
| 5 | Methacrylic acid copolymer | 5-50 |
| 6 | Purified water | q.s. |

Blending:

| | | |
|---|---|---|
| 1 | MR1 Component | — |
| 2 | MR2 Component | — |
| 3 | Micronized talc | 0.1-5 |

### Process:

### MR1 Component:

Salsalate, microcrystalline cellulose and croscarmellose sodium are mixed and granulated with a dispersion of hypromellose in purified water. The granules are extruded and spheronized to yield pellets. The MR1 pellets are dried to provide immediate release pellets of salsalate.

### MR2 Component:

Salsalate, microcrystalline cellulose and croscarmellose sodium are mixed and granulated with a dispersion of hypromellose in purified water. The granules are extruded and spheronized to yield pellets. The pellets are dried and seal coated with a dispersion of hypromellose in purified water. The coated MR2 pellets are coated with a dispersion of methacrylic acid copolymer to provide modified release pellets of salsalate.

### Capsules:

The MR1 pellets and MR2 pellets are mixed together along with micronized talc and filled in appropriate sized capsules.

In an alternative embodiment to Example 4, the capsules may be further coated with a modified release coating.

### Example 7

| | | |
|---|---|---|
| | | Quantity |
| Sr. No. | Ingredients | (% w/w) |

MR1 Component:

| | | |
|---|---|---|
| 1 | Salsalate | 30-50 |
| 2 | Microcrystalline Cellulose | 5-20 |
| 3 | Croscarmellose Sodium | 0.5-10 |
| 4 | Hypromellose | 0.5-10 |
| 5 | Methacrylic Acid Copolymer | 5-50 |
| 6 | Purified Water | q.s. |

MR2 Component:

| | | |
|---|---|---|
| 1 | Salsalate | 20-40 |
| 2 | Microcrystalline Cellulose | 4-10 |
| 3 | Croscarmellose Sodium | 0.5-10 |
| 4 | Hypromellose | 0.1-10 |
| 5 | Purified Water | q.s. |

Blending:

| | | |
|---|---|---|
| 1 | MR2 Component | q.s. |
| 2 | MR1 Component | q.s. |
| 3 | Stearic Acid | 0.1-5 |

Film Coating:

| | | |
|---|---|---|
| 1 | Opadry White | 0.5-5 |
| 2 | Purified Water | q.s. |

### Process:

### MR1 Component:

Salsalate, microcrystalline cellulose and croscarmellose sodium are mixed and granulated with a dispersion of hypromellose in purified water. The granules are extruded and spheronized to yield pellets. The pellets are dried and seal coated with a dispersion of hypromellose in purified water. The coated MR1 pellets are coated with a dispersion of methacrylic acid copolymer to provide modified release pellets of salsalate.

### MR2 Component:

Salsalate, microcrystalline cellulose and croscarmellose sodium are mixed and granulated with a dispersion of hypromellose in purified water. The MR2 granules are dried to provide immediate release granules of salsalate.

### Tablets:

The MR1 pellets and MR2 granules are mixed together along with stearic acid and compressed using appropriate tooling to yield tablets. These tablets are then film coated.

In an alternative embodiment to Example 5, the tablets may be further coated with a modified release coating.

### Example 8

| | | |
|---|---|---|
| | | Quantity |
| Sr. No. | Ingredients | (% w/w) |

MR1 Component:

| | | |
|---|---|---|
| 1 | Salsalate | 20-50 |
| 2 | Microcrystalline Cellulose | 5-30 |
| 3 | Croscarmellose Sodium | 0.5-10 |
| 4 | Hypromellose | 0.1-10 |
| 5 | Purified Water | q.s. |

MR2 Component:

| | | |
|---|---|---|
| 1 | Salsalate | 10-30 |
| 2 | Microcrystalline Cellulose | 1-20 |
| 3 | Croscarmellose Sodium | 0.1-5 |
| 4 | Hypromellose | 0.1-5 |
| 5 | Methacrylic Acid Copolymer | 1-20 |
| 6 | Purified Water | q.s. |

MR3 Component:

| | | |
|---|---|---|
| 1 | Salsalate | 10-30 |
| 2 | Microcrystalline Cellulose | 1-10 |
| 3 | Croscarmellose Sodium | 0.1-2 |
| 4 | Hypromellose | 0.1-10 |
| 5 | Methacrylic Acid Copolymer | 1-30 |
| 6 | Triethyl Citrate | 0.5-5 |
| 7 | Micronized Talc | 0.5-5 |
| 8 | Purified Water | q.s. |

Blending:

| | | |
|---|---|---|
| 1 | MR1 Component | q.s. |
| 2 | MR2 Component | q.s. |
| 3 | MR3 Component | q.s. |
| 4 | Micronized Talc | 0.1-5 |

### Process:

### MR1 Component

Salsalate, microcrystalline cellulose and croscarmellose sodium are mixed and granulated with a dispersion of hypromellose in purified water. The granules are extruded and spheronized to yield pellets. The MR1 pellets are dried to provide immediate release pellets of salsalate.

### MR2 Component:

Salsalate, microcrystalline cellulose and croscarmellose sodium are mixed and granulated with a dispersion of hypromellose in purified water. The granules are extruded and spheronized to yield pellets. The pellets are dried and seal coated with a dispersion of hypromellose in purified water. The coated MR2 pellets are coated with a dispersion of methacrylic acid copolymer to provide modified release pellets of salsalate.

### MR3 Component:

Salsalate, microcrystalline cellulose and croscarmellose sodium are mixed and granulated with a dispersion of hypromellose in purified water. The granules are extruded and spheronized to yield pellets. The pellets are dried and seal coated with a dispersion of hypromellose in purified water. The coated MR3 pellets are coated with a dispersion of a different methacrylic acid copolymer than used in the MR2 component to provide modified release pellets of salsalate with a different release profile than of the MR2 pellets.

### Capsules:

The MR1 pellets, MR2 pellets and MR3 pellets are mixed together along with micronized talc and filled in appropriate sized capsules.

In alternative embodiment to the capsules of Example 6, the capsules may be coated with a modified release coating.

### Example 10

| | | |
|---|---|---|
| | | Quantity |
| Sr. No. | Ingredients | (% w/w) |

MR1 Component:

| | | |
|---|---|---|
| 1 | Salsalate | 10-30 |
| 2 | Microcrystalline Cellulose | 1-10 |
| 3 | Croscarmellose Sodium | 0.1-10 |
| 4 | Hypromellose | 0.1-10 |
| 5 | Methacrylic Acid Copolymer | 1-20 |
| 6 | Purified Water | q.s. |

MR2 Component:

| | | |
|---|---|---|
| 1 | Salsalate | 10-30 |
| 2 | Microcrystalline Cellulose | 1-10 |
| 3 | Croscarmellose Sodium | 0.1-10 |
| 4 | Hypromellose | 0.1-10 |
| 5 | Methacrylic Acid Copolymer | 1-20 |
| 6 | Triethyl Citrate | 0.5-5 |
| 7 | Micronized Talc | 0.5-5 |
| 8 | Purified Water | q.s. |

MR3 Component:

| | | |
|---|---|---|
| 1 | Salsalate | 10-30 |
| 2 | Microcrystalline Cellulose | 1-10 |
| 3 | Croscarmellose Sodium | 0.1-10 |
| 4 | Hypromellose | 0.1-10 |
| 5 | Purified Water | q.s. |

Blending:

| | | |
|---|---|---|
| 1 | MR3 Component | q.s. |
| 2 | MR1 Component | q.s. |
| 3 | MR2 Component | q.s. |
| 4 | Stearic Acid | 0.1-5 |

Film Coating:

| | | |
|---|---|---|
| 1 | Opadry White | 0.5-5 |
| 2 | Purified Water | q.s. |

### Process:

### MR1 Component:

Salsalate, microcrystalline cellulose and croscarmellose sodium are mixed and granulated with a dispersion of hypromellose in purified water. The granules are extruded and spheronized to yield pellets. The pellets are dried and seal coated with a dispersion of hypromellose in purified water. The coated MR1 pellets are coated with a dispersion of methacrylic acid copolymer to provide modified release pellets of salsalate.

### MR2 Component:

Salsalate, microcrystalline cellulose and croscarmellose sodium are mixed and granulated with a dispersion of hypromellose in purified water. The granules are extruded and spheronized to yield pellets. The pellets are dried and seal coated with a dispersion of hypromellose in purified water. The coated MR2 pellets are coated with a dispersion of different type of methacrylic acid copolymer than used in the MR1 pellets to provide modified release pellets of salsalate with a different release profile than that of the MR1 pellets.

### MR3 Component:

Salsalate, microcrystalline cellulose and croscarmellose sodium are mixed and granulated with a dispersion of hypromellose in purified water. The granules are dried to provide immediate release MR3 granules of salsalate.

### Tablets:

The MR1 pellets, MR2 pellets and MR3 granules are mixed together along with stearic acid and compressed using appropriate tooling to yield tablets. These tablets are film coated.

In an alternative embodiment to Example 9, the tablets may be further coated with a modified release coating.

### Example 10

### Salsalate Modified Release Tablets 750 mg (Fasting Study) Pharmacokinetic Summary

A randomized, two-way, crossover study was conducted in 12 healthy adult human male volunteers out of whom 11 volunteers completed the study. The volunteers were administered one Salsalate Modified Release Tablets 750 mg (per Example 2) and Salsalate Tablets, USP 750 mg under fasted conditions in a crossover manner. The pharmacokinetic parameters of the parent compound (salsalate) and active metabolite (salicylic Acid) are summarized in below:

### Salsalate:

Salsalate

| | Salsalate Modified | Salsalate Tablets, USP |
|---|---|---|
| Pharmacokinetic | Release Tablets (750 mg) | 750 mg |
| Parameters | Mean ± SD | Mean ± SD |
| Cmax (μg/ml) | 19.24 ± 10.26 | 18.62 ± 3.59 |
| AUCt (μg * hr/ml) | 41.76 ± 19.68 | 48.14 ± 8.98 |
| AUCi (μg * hr/ml) | 46.04 ± 15.26 | 48.37 ± 8.98 |
| Tmax^{#} (hr) | 4.00 (2.00, 8.00) | 2.00 (1.50, 3.00) |
| Thalf (hr) | 1.05 ± 0.30 | 0.91 ± 0.22 |

^{#}Median (Range)

Compared to the immediate release formulation, there is little difference in mean Cmax value for the modified release formulation. However, the mean AUC₀₋ₜ and AUC_{0-inf} is decreased by approximately 13% and 5%, respectively in the modified release formulation. The median time-to-peak concentration (Tmax) is also delayed by 2.00 hours in the modified release formulation compared to the immediate release formulation (4.00 hours versus 2.00 hours). The mean half life of the immediate release and modified release formulations are very similar for salsalate.

Salicylic Acid

| | Salsalate Modified | Salsalate Tablets, USP |
|---|---|---|
| Pharmacokinetic | Release Tablets (750 mg) | 750 mg |
| Parameters | Mean ± SD | Mean ± SD |
| Cmax (μg/ml) | 38.77 ± 17.47 | 51.63 ± 7.56 |
| AUCt (μg * hr/ml) | 32.41 ± 17.11 | 440.22 ± 114.25 |
| AUCi (μg * hr/ml) | 333.36 ± 178.11 | 448.46 ± 122.52 |
| Tmax^{#} (hr) | 5.50 (3.50, 9.00) | 4.50 (3.00, 6.00) |
| Thalf (hr) | 2.61 ± 0.87 | 2.57 ± 0.81 |

^{#}Median (Range)

Compared to the immediate release formulation, the mean Cmax is decreased by 25% and both mean AUG₀₋ₜ and AUG_{0-inf} is decreased by approximately 26% in the modified release formulation. The median time-to-peak concentration (Tmax) is also delayed by 1.00 hour in the modified release formulation as compared to the immediate release formulation (5.50 hours versus 4.50 hours). The mean half life of the immediate release and modified release formulations are very similar for salicylic acid.

### Example 11

### Calculation of dC/dT for Salsalate and Salicylic Acid

The change in concentration over time for salsalate and salicylic acid upon administration of 750 mg modified release salsalate tablets (per Example 2) are compared against 750 mg immediate release salsalate tablets. The results are provided below:

dC/dT Calculation-Salsalate

| | | | | | % | |
|---|---|---|---|---|---|---|
| | Mean Concentration | | | | Difference | % |
| Time | (μg/mL) | | dC/dT | | for | Difference |
| (hr) | Test | Reference | Test | Reference | reference | for Test |
| 0 | 0.000 | 0.000 | — | — | — | |
| 0.5 | 0.000 | 2.837 | 0.000 | 5.674 | 100.000 | 0 |
| 1 | 0.000 | 8.898 | 0.000 | 8.898 | 100.000 | 0 |
| 1.5 | 2.826 | 12.187 | 1.884 | 8.125 | 76.811 | 23.19 |
| 2 | 6.501 | 14.982 | 3.251 | 7.491 | 56.608 | 43.39 |
| 2.5 | 6.567 | 15.199 | 2.627 | 6.080 | 56.793 | 43.21 |
| 3 | 7.139 | 13.538 | 2.380 | 4.513 | 47.267 | 52.73 |
| 3.5 | 11.097 | 10.095 | 3.171 | 2.884 | −9.926 | 109.93 |
| 4 | 9.935 | 6.984 | 2.484 | 1.746 | −42.254 | 142.25 |
| 4.5 | 8.008 | 4.647 | 1.780 | 1.033 | −72.326 | 172.33 |
| 5 | 6.896 | 2.560 | 1.379 | 0.512 | −169.375 | 269.38 |
| 5.5 | 6.657 | 1.535 | 1.210 | 0.279 | −333.681 | 433.68 |
| 6 | 5.158 | 0.977 | 0.860 | 0.163 | −427.943 | 527.94 |
| 6.5 | 3.900 | 0.653 | 0.600 | 0.100 | −497.243 | 597.24 |
| 7 | 2.961 | 0.442 | 0.423 | 0.063 | −569.910 | 669.91 |
| 7.5 | 1.957 | 0.328 | 0.261 | 0.044 | −496.646 | 596.65 |
| 8 | 1.368 | 0.217 | 0.171 | 0.027 | −530.415 | 630.41 |
| 9 | 0.556 | 0.067 | 0.062 | 0.007 | −729.851 | 829.85 |
| 10 | 0.262 | 0.029 | 0.026 | 0.003 | −803.448 | 903.45 |
| 11 | 0.120 | 0.018 | 0.011 | 0.002 | −566.667 | 666.67 |
| 12 | 0.050 | 0.013 | 0.004 | 0.001 | −284.615 | 384.62 |
| 14 | 0.018 | 0.000 | 0.001 | 0.000 | — | |
| 16 | 0.000 | 0.000 | 0.000 | 0.000 | — | |
| 18 | 0.000 | 0.000 | 0.000 | 0.000 | — | |
| 24 | 0.000 | 0.000 | 0.000 | 0.000 | — | |
| 48 | 0.000 | 0.000 | 0.000 | 0.000 | — | |
| 96 | 0.000 | 0.000 | 0.000 | 0.000 | — | |

The data above demonstrates that in a single dose human pharmacokinetic study of a modified release composition of salsalate, the composition provides a change in plasma concentration of salsalate as a function of time (dC/dT) over a defined period between 0 to 3 hours after administration that is less than about 65% of the dC/dT of the same quantity of an immediate release form of salsalate over the defined time period.

dC/dT Calculation-Salicylic Acid

| | | | | | % | |
|---|---|---|---|---|---|---|
| | | | | | Differ- | |
| | | | | | ence | % |
| | Mean Concentration | | | | for | Differ- |
| Time | (μg/mL) | | dC/dT | | refer- | ence |
| (hr) | Test | Reference | Test | Reference | ence | for Test |
| 0.00 | 0.000 | 0.000 | — | — | — | |
| 0.50 | 0.000 | 1634.182 | 0.000 | 3268.364 | 100.000 | 0.00 |
| 1.00 | 0.000 | 7486.364 | 0.000 | 7486.364 | 100.000 | 0.00 |
| 1.50 | 1024.545 | 15065.455 | 683.030 | 10043.637 | 93.199 | 6.80 |
| 2.00 | 5032.000 | 24617.273 | 2516.000 | 12308.637 | 79.559 | 20.44 |
| 2.50 | 9329.182 | 33454.545 | 3731.673 | 13381.818 | 72.114 | 27.89 |
| 3.00 | 12624.091 | 40981.818 | 4208.030 | 13660.606 | 69.196 | 30.80 |
| 3.50 | 18877.364 | 46145.455 | 5393.533 | 13184.416 | 59.092 | 40.91 |
| 4.00 | 24051.818 | 48318.182 | 6012.955 | 12079.546 | 50.222 | 49.78 |
| 4.50 | 29324.364 | 51354.545 | 6516.525 | 11412.121 | 42.898 | 57.10 |
| 5.00 | 29810.000 | 46927.273 | 5962.000 | 9385.455 | 36.476 | 63.52 |
| 5.50 | 31605.000 | 44900.000 | 5746.364 | 8163.636 | 29.610 | 70.39 |
| 6.00 | 32510.727 | 42436.364 | 5418.455 | 7072.727 | 23.389 | 76.61 |
| 6.50 | 32445.909 | 40754.545 | 4991.678 | 6269.930 | 20.387 | 79.61 |
| 7.00 | 32532.727 | 38045.455 | 4647.532 | 5435.065 | 14.490 | 85.51 |
| 7.50 | 31471.818 | 35700.000 | 4196.242 | 4760.000 | 11.844 | 88.16 |
| 8.00 | 29852.727 | 32900.000 | 3731.591 | 4112.500 | 9.262 | 90.74 |
| 9.00 | 27007.273 | 28445.455 | 3000.808 | 3160.606 | 5.056 | 94.94 |
| 10.00 | 23739.909 | 24827.273 | 2373.991 | 2482.727 | 4.380 | 95.62 |
| 11.00 | 20258.091 | 20715.455 | 1841.645 | 1883.223 | 2.208 | 97.79 |
| 12.00 | 17468.909 | 17312.727 | 1455.742 | 1442.727 | −0.902 | 100.90 |
| 14.00 | 11688.182 | 11523.636 | 834.870 | 823.117 | −1.428 | 101.43 |
| 16.00 | 7642.636 | 7298.182 | 477.665 | 456.136 | −4.720 | 104.72 |
| 18.00 | 4729.909 | 4582.727 | 262.773 | 254.596 | −3.212 | 103.21 |
| 24.00 | 1176.182 | 1029.000 | 49.008 | 42.875 | −14.303 | 114.30 |
| 48.00 | 0.000 | 0.000 | 0.000 | 0.000 | — | |
| 96.00 | 0.000 | 0.000 | 0.000 | 0.000 | — | |

The data above demonstrates that in a single dose human pharmacokinetic study of a modified release composition of salsalate, the composition provides a change in plasma concentration of salicylic acid as a function of time (dC/dT) over a defined period between 0 to 4 hours after administration that is less than about 65% of the dC/dT of the same quantity of an immediate release form of salsalate over the defined time period.

While the invention has been described in terms of its specific embodiments, certain modifications and equivalents will be apparent to those skilled in the art and are intended to be included within the scope of the invention.

1. A modified release pharmaceutical composition comprising salsalate or a pharmaceutically acceptable salt thereof, wherein the composition releases substantially no drug within the first two hours of its administration.

2. The modified release pharmaceutical composition of claim 1, wherein the composition comprises about 100 mg to about 1000 mg of salsalate and provides an in-vivo plasma profile for salsalate when administered in a fasted state comprising:
a mean of Cₘₐₓ less than about 30 μg/mL,
a mean of AUC_{0-∞} less than about 60 μg*hr/mL; and
a mean of Tₘₐₓ at least about 2 hours.

3. The modified release pharmaceutical composition of claim 1, wherein the composition comprises about 100 mg to about 1000 mg of salsalate and provides an in-vivo plasma profile for salicylic acid when administered in a fasted state comprising:
a mean of Cₘₐₓ less than about 55 μg/mL,
a mean of AUC_{0-∞} less than about 500 μg*hr/mL; and
a mean of Tₘₐₓ at least about 4.5 hours.

4. The modified release pharmaceutical composition of claim 1, wherein the salsalate is present in an amount of at least 50% by weight of the total composition.

5. The modified release pharmaceutical composition according to claim 4, wherein the total weight of the composition is between 900 mg to 1500 mg, when the amount of salsalate in the composition is about 750 mg and between 550 mg to 1000 mg, when the amount of salsalate in the composition is about 500 mg.

6. The modified release pharmaceutical composition according to claim 1, wherein the composition consists essentially of salsalate as the active ingredient and one or more enteric polymers and one or more pharmaceutically acceptable excipients.

7. The modified release pharmaceutical composition according to claim 6, wherein the enteric polymer is in a matrix with salsalate or coated on a core comprising salsalate.

8. The modified release pharmaceutical composition according to claim 6, wherein the composition further comprises an additional top coat of one or more flavoring agents.

9. The modified release pharmaceutical composition according to claim 6, wherein the composition is in the form of a tablet, a capsule, granules, powder, pellets, minitablets, microtablets, or a sachet.

10. The modified release pharmaceutical composition according to claim 6, wherein the pharmaceutically acceptable excipients comprise one or more of diluents, disintegrants, binders, stabilizers, buffering agents, lubricants, glidants, antiadherents, solubilizers, taste-masking agents, sweeteners, flavoring agents, and solvents.

11. The modified release pharmaceutical composition according to claim 6, wherein the enteric polymer comprises one or more of hydroxypropyl methylcellulose phthalate, cellulose acetate phthalate, cellulose acetate succinate, methylcellulose phthalate, hydroxypropyl methylcellulose phthalate, ethylhydroxycellulose phthalate, polyvinylacetate phthalate, polyvinyl butyrate acetate, vinyl acetate-maleic anhydride copolymer, styrene-maleic mono-ester copolymer, carboxymethyl ethylcellulose, methyl methacrylate-methacrylic acid copolymer (Eudragit L-100 (methacrylic acid copolymer L) or Eudragit S-100 (methacrylic acid copolymer S)), methacrylic acid-ethyl acrylate copolymer (Eudragit L100-55 (dried methacrylic acid copolymer LD) or Eudragit L30D-(methacrylic acid copolymer LD)), methacrylic acid-methyl acrylate-methyl methacrylate copolymer (Eudragit FS30D), hydroxypropyl cellulose acetate succinate (HPMCAS), and shellac.

12. The modified release pharmaceutical composition according to claim 6, wherein the composition retains at least about 80% of the potency of salsalate in the pharmaceutical composition after storage for three months at 40° C. and 75% relative humidity.

13. The modified release pharmaceutical composition according to claim 6, wherein the composition exhibits an in vitro dissolution profile, when measured in a USP dissolution apparatus type I, at 150 rpm, at a temperature of 37.0±0.5° C. in 900 ml of 0.1 N HCl for first two hours followed by measurement in pH 7.4 phosphate buffer, such that at most 10% of salsalate is released in the first two hours and at least 80% of salsalate is released within the next two hours.

14. The pharmaceutical composition according to claim 1, wherein the composition provides a change in plasma concentration of salsalate as a function of time (dC/dT) over a defined period between 0 and 3 hours after administration that is less than about 65% of the dC/dT of the same quantity of an immediate release form of salsalate over said defined time period, wherein the dC/dT is measured in a single dose human pharmacokinetic study.

15. The pharmaceutical composition according to claim 1, wherein the composition provides a change in plasma concentration of salicylic acid as a function of time (dC/dT) over a defined period between 0 and 4 hours after administration that is less than about 65% of the dC/dT of the same quantity of an immediate release form of salsalate over said defined time period, wherein the dC/dT is measured in a single dose human pharmacokinetic study.

16. The modified release pharmaceutical composition according to claim 1 made by a process comprising:
i. mixing and/or granulating salsalate, one or more enteric polymers and one or more pharmaceutically acceptable excipients;
ii. compressing the mixture or granules to form a tablet; and
iii. optionally coating the tablet.

17. The modified release pharmaceutical composition according to claim 1 made by a process comprising:
i. preparing a core comprising salsalate and one or more pharmaceutically acceptable excipients;
ii. optionally coating the core with an intermediate layer; and
iii. coating the core of step (i) or product of step (ii) with a layer comprising one or more enteric polymers.

18. The modified release pharmaceutical composition according to claim 1 made by a process comprising:
i. preparing an inert core;
ii. coating the inert core with a solution/suspension comprising salsalate and one or more pharmaceutically acceptable excipients;
iii. coating the drug layered core of step (ii) with one or more enteric layers; and
iv. optionally coating the product of step (iii) with a functional/non-functional layer.

19. The modified release pharmaceutical composition according to claim 1 made by a process comprising:
i. mixing and/or granulating salsalate with one or more pharmaceutically acceptable excipients;
ii. filling the mixture or granules into a capsule; and
iii. coating the capsule with an enteric coating.

20. A method for providing relief of the signs and symptoms of rheumatoid arthritis, osteoarthritis and related rheumatic disorder in a patient in need thereof, comprising administering to the patient about 100 mg to about 1000 mg of salsalate in one or more modified release oral dosage forms, wherein the administering step provides a mean maximum plasma concentration (Cₘₐₓ) less than 30 μg/mL of salsalate, a mean AUC_{0-∞} less than about 60 μg*hr/mL and a mean Tₘₐₓ of at least 2 hours to the patient.

21. The method of claim 20, wherein at steady state the composition has a fluctuation index about 5-30% of a fluctuation index achieved with an immediate-release composition of the salsalate.

22. The method for claim 20, wherein the incidences of side effects associated with salsalate administration in immediate release form at the same strength is reduced.

23. A modified release pharmaceutical composition consisting essentially of 750 mg of salsalate or a pharmaceutically acceptable salt thereof as the only active ingredient in the composition, an enteric polymer to control the release of the salsalate from the composition based on a change in pH, and one or more pharmaceutical excipients, wherein the composition exhibits an in vitro dissolution profile, when measured in a USP dissolution apparatus type I, at 150 rpm, at a temperature of 37.0±0.5° C. in 900 ml of 0.1 N HCl for the first two hours followed by measurement in pH 7.4 phosphate buffer, such that at most 10% of salsalate is released in the first two hours and at least 80% of salsalate is released within the next two hours.
